(19) United States
(12) Patent Application Publication
(10) Pub. No.: US 2017/0119834 A1
(43) Pub. Date: May 4, 2017

(54) Methods of Treating Diarrhea in Adult Non-Human Animals

(71) Applicant: Jaguar Animal Health, Inc., San Francisco, CA (US)

(72) Inventor: Serge Martinod, Groton, CT (US)

(73) Assignee: Jaguar Animal Health, Inc., San Francisco, CA (US)

(21) Appl. No.: 15/314,171

(22) PCT Filed: May 28, 2015
(86) PCT No.: PCT/US2015/032920

§ 371 Date: Nov. 28, 2016

Related U.S. Application Data
(60) Provisional application No. 62/087,207, filed on Dec. 3, 2014.
(60) Provisional application No. 62/004,650, filed on May 29, 2014.

(51) Int. Cl. A61K 36/47; A61K 9/00; A61K 9/06; A61K 31/353

(52) U.S. Cl. CPC A61K 36/47; A61K 31/353; A61K 9/0095; A61K 9/0056; A61K 9/06

(57) ABSTRACT

Methods of treating adult non-human animals afflicted with diarrhea, particularly, stress-induced diarrhea, by administering to an animal in need thereof an effective amount of a pharmaceutical formulation of a proanthocyanidin polymer composition isolated from a Croton spp. or a Calophyllum spp. are provided. In particular, the adult animals include horses and camels which frequently suffer from stress-induced diarrhea and its symptoms, and the administered proanthocyanidin polymer composition is isolated from a Croton lechleri. The composition, which can be enteric or non-enteric, is administered particularly to target the intestine/gut of the animal via a mode and under conditions conducive and sufficient for providing effective treatment of diarrhea in the affected animal. Preferred modes of administration include orally providing an effective concentration of the proanthocyanidin polymer composition, e.g., via an oral paste formulation, drench, animal feed, or water bottle, to the affected animal.

### FIELD OF THE INVENTION

The invention relates to the treatment of diarrhea in adult animals with a proanthocyandin polymeric composition isolated from the plant Croton spp. or Calophyllum spp., or with a latex, extract, or food supplement derived therefrom. More particularly, the composition is effective in treating secretory or watery diarrhea of various etiologies, especially stress-induced diarrhea, and reducing the severity and duration of diarrhea in afflicted adult animals, as well as increasing their survivability.

### BACKGROUND OF THE INVENTION

A wide array of infectious and pathogenic agents including bacteria, viruses, and parasites cause diarrhea in non-human animals, particularly, domesticated livestock animals associated with farming, food, and labor. Many of these enteropathogens cause one or more adverse effects in the animals, such as severe intestinal lesions, dehydration, alterations in enzyme activity, and/or alterations in nutrient transport mechanisms. The clinical presentation of diarrhea caused by these agents may vary; some diarrheas are self-limiting, while others are associated with high morbidity or high mortality (R. E. Holland, 1990, Clin. Microbiol. Rev., 3(4):345-375).

Secretory diarrheas, also called watery diarrheas, are a major source of illness and mortality in adult, non-human animals and are characterized by the loss of both fluid and electrolytes through the intestinal tract, leading to serious and often life-threatening dehydration. Secretory diarrhea is caused by a variety of bacterial, viral and protozoal pathogens and also results from other non-infectious etiologies such as ulcerative colitis, inflammatory bowel disease, environmental and stress conditions, and cancers and neoplasias of the gastrointestinal tract. All types of diarrheal disease may have a secretory component.

Vibrio cholerae and Escherichia coli are the major bacterial causative agents of secretory diarrhea. The enterotoxigenic, enterohemorrhagic, enteroinvasive, and enteropathogenic strains of E. coli represent important sources of secretory diarrhea. Bacterial microorganisms which cause secretory diarrhea include Vibrio spp., Campylobacter spp., Salmonella spp., Aeromonas spp., Plesiomonas spp., Shigella spp., Klebsiella spp., Citrobacter spp., Yersinia spp., Clostridium spp., Bacteroides spp., Staphylococcus spp., and Bacillus spp., as well as other enteric bacteria. Secretory diarrhea can also be caused by protozoal pathogens such as Cryptosporidium spp., for example Cryptosporidium parvum. See generally, Holland, 1990, Clin. Microbiol. Rev. 3:345; Harris, 1988, Ann. Clin. Lab. Sci. 18:102; Gracey, 1986, Clin. in Gastroent., 15:21; Ooms and Degryse, 1986, Veterinary Res. Comm. 10:355; Black, 1982, Med. Clin. Nor. Am., 66:611.

V. cholerae, the enterotoxigenic strains of E. coli, such as K99, and a variety of other enteric bacteria elicit secretory diarrhea via similar mechanisms. These pathogens produce a toxin which binds a specific receptor on the apical membrane of the intestinal epithelium. Binding of the receptor triggers an adenylate cyclase- or guanylate cyclase-mediated signal transduction leading to an increase in cAMP or cGMP. This regulatory cascade, apparently acting through phosphorylation of specific apical membrane proteins, stimulates chloride efflux into the gut from the intestinal epithelial crypt cells and inhibits normal resorption of sodium and chloride ions by the intestinal epithelial villus cells. The increased chloride and sodium ion concentration osmotically draws water into the intestinal lumen, resulting in both dehydration and loss of electrolytes in the afflicted organism. Agents which reduce chloride ion secretion will, therefore, prevent the fluid movement into the intestine and resulting net fluid elimination. Thus, such agents are particularly useful for treating and preventing the dangerous dehydration and electrolyte loss associated with secretory diarrhea.

The available and commonly used treatments for diarrhea in adult non-human animals typically involve vital fluid replacement and electrolyte replenishment to counter or stop fluid and electrolyte loss. Other types of treatments include the administration of gut-lining protectants, e.g., bismuth, oral antibiotics, and agents that affect gut motility. Depending on the cause(s), timing, severity and course of diarrhea and/or its associated disease or condition, the various known treatments may or may not be effective, and the animals may or may not respond adequately or in a timely manner, leading to premature death in many cases. Because the economic and humane impacts of diarrhea and its related conditions on the afflicted animals, their handlers and caregivers are so great, there is a compelling need for alternative, safe, and medically effective, as well as cost effective, treatments and remedies for diarrhea and its associated symptoms in non-human adult animals. The present invention addresses such a need.

### SUMMARY OF THE INVENTION

The present invention relates to methods of treating diarrhea, particularly stressed-induced diarrhea, in adult non-human animals in need thereof by administering a polymeric proanthocyanidin, i.e., a proanthocyanidin polymer, from a Croton species or Calophyllum species. In an embodiment, a pharmaceutically or physiologically acceptable formulation or composition comprising a proanthocyanidin polymer from a Croton species or Calophyllum species is administered. In particular embodiments, a proanthocyanidin polymer from Croton lechleri, or pharmaceutically acceptable formulation or composition comprising a proanthocyanidin polymer from Croton lechleri is administered.

In an embodiment, the proanthocyanidin polymer composition is a latex or extract from a Croton species or Calophyllum species, in particular, Croton lechleri. In another embodiment, the composition is a botanical extract of Croton lechleri containing a proanthocyanidin oligomer, or a food supplement formulation of the botanical extract of Croton lechleri. Such Croton species or Calophyllum species latex or extract compositions can be more highly purified as described herein. In an embodiment, the methods involve the administration of a pharmaceutically acceptable composition comprising a proanthocyanidin polymer from Croton lechleri to an animal in need thereof. In an embodiment, the methods involve the administration of a proanthocyanidin polymer from Croton lechleri, or a pharmaceutically acceptable composition comprising a proanthocyanidin polymer from Croton lechleri, wherein the proanthocyanidin polymer or oligomer from C. lechleri is also known as crofelemer (a purified proanthocyanidin oligomer), SP 303, SB 300, as further described herein. In certain embodiments, the C. lechleri proanthocyanidin polymer, or composition thereof, is in an enteric coated form. In other embodiments, the C. lechleri proanthocyanidin polymer, or composition thereof, is in a non-enteric coated form.

The invention provides a method of treating and preventing the debilitating effects of stress-induced diarrhea in adult non-human animals. In particular, the methods treat and prevent dehydration associated with water, fluid and electrolyte losses in animals afflicted with diarrhea. In other embodiments, the methods treat and prevent diarrhea associated with colitis, including acute colitis, in afflicted animals. Thus, in an embodiment, the methods of the invention provide antisecretory treatments for diarrhea, particularly, secretory or watery diarrhea, in adult non-human animals.

The invention is also more particularly directed to a method of treating, preventing, ameliorating or controlling stress-induced diarrhea in adult equine or camel species in need thereof by administering a proanthocyanidin polymer from Croton lechleri in an effective amount to control or treat the diarrhea in these animals. In particular embodiments the adult equine or camel species are racing horses or camels. The invention includes treatment, prevention or amelioration of the symptoms of stress-induced diarrhea in any adult non-human animal subjected to stress due to demanding performance requirements—such as racing. In an embodiment the proanthocyanidin polymer is a formulation, composition, or extract from Croton lechleri. In an embodiment, the proanthocyanidin polymer from Croton lechleri is a more highly purified composition, also termed crofelemer or SB 300 compositions herein.

The invention is also more particularly directed to a method of improving gut health, controlling diarrhea and normalizing stool formation in adult, non-human animals in need thereof by administering a proanthocyanidin polymer from Croton lechleri in an effective amount to control or treat the diarrhea in these animals. In an embodiment the proanthocyanidin polymer is a formulation, composition, or botanical extract from Croton lechleri. In an embodiment, the formulation, composition, or botanical extract from Croton lechleri is in the form of a paste or gel. In a particular embodiment, the paste formulation comprises beads (nano or microparticles) comprising enterically coated SB 300 or SP 303 and is orally administered to an animal in need. In an embodiment, the paste formulation comprises beads (nano or microparticles) comprising enterically coated SB 300. In a particular embodiment, the paste comprising SB 300 enteric beads is orally administered to an animal in need twice daily for three days. In some embodiments, the paste is orally administered for three consecutive days. In an embodiment, the paste comprising SB 300 enteric beads is orally administered to an animal in need at a dose of 2 mg/kg twice daily for three days.

Secretory diarrhea is a significant problem in adult animals, including large-sized animals prone to stress as well as other causative factors of diarrheal disease. Stress-induced diarrhea is a common, yet frequently overlooked condition that can debilitate adult animals, particularly those involved in the sports or racing industry throughout the world, including the United States, the United Arab Emirates, Saudi Arabia, China, India and the like. In particular, stress-induced diarrhea affects a large number of animals, such as horses and camels, that participate in the sport of racing in the U.S. and the rest of the world. By way of example, the sport of horse and camel racing can involve hundreds of thousands to millions of animals per year, worldwide.

Diarrheal disease in farm animals, particularly food or livestock animals such as cattle, steer, sheep and swine, is often caused by bacterial pathogens such as enterotoxigenic, enterohemorrhagic microorganisms, including, without limitation, E. coli, Salmonella spp., Clostridium perfringens, Bacteroides fragilis, Campylobacter spp., and Yersinia enterocolitica. In addition, protozoal pathogens, particularly Cryptosporidium parvum, and viruses, particularly rotaviruses and coronaviruses, are significant causes of diarrhea in farm animals. Other viruses have been implicated as causative agents of diarrhea in farm animals and include togaviruses, parvoviruses, caliciviruses, adenoviruses, bredaviruses, and astroviruses. See, generally, Holland, 1990, Clin. Microbiology Rev. 3:345; see also Gutzwiller and Blum, 1996, AJVR 57:560; Strombeck, 1995, Veterinary Quarterly 17(Suppl. 1):512; Vermunt, 1994, Austral. Veterinary J. 71:33; Driesen et al., 1993, Austral. Veterinary J. 70:259; Mouricout, 1991, Eur. J. Epidemiol. 7:588; Ooms and Degryse, 1986, Veterinary Res. Comm. 10:355.

The invention provides treatment methods effective for reducing and/or alleviating diarrhea and/or its associated symptoms in adult non-human animals in need thereof. In particular, the methods are directed to the treatment of diarrhea, particularly secretory/watery diarrhea, which may be caused by a variety of etiological agents and/or environmental factors. In a particular embodiment, the methods of the invention treat diarrhea caused by stress in the adult, non-human animals. The invention further provides formulations and compositions suitable for treating diarrhea in adult non-human animals. In some embodiments, the diarrhea is episodic diarrhea. The non-human animal species suitable for treatment by the practice of the methods of the present invention are not intended to be limiting, and can include, for example, farm and livestock animals in the bovine, swine, ovine, camel and equine families, as well as other domestic, non-human animals such as canine animals and feline animals. Unless otherwise noted herein, use of the term “animal” herein denotes non-human, warm-blooded mammals of a number of different species.

Provided by the invention are methods of treating, preventing, or reducing the incidence or severity of diarrhea, particularly, stress-induced diarrhea, in adult equine animals. Also provided by the invention are methods of treating, preventing or reducing the incidence or severity of diarrhea, particularly, stress-induced diarrhea, in adult camels. In an embodiment, the stress-induced diarrhea can also result from breeding and foaling of equine animals and camels, as well as the practices and conditions related thereto.

The present invention further provides a method of treating, preventing, ameliorating the symptoms of or reducing the incidence or severity of stress-induced diarrhea and/or its associated symptoms in an adult non-human animal, the method comprising orally administering to an animal in need thereof a pharmaceutically acceptable composition comprising an aqueous soluble proanthocyanidin polymer from Croton lechleri, wherein the composition is administered under conditions to provide an effective amount of the composition in the stomach/gut/intestine of the adult animal, thereby treating, preventing, ameliorating, the symptoms of or reducing the incidence or severity of the diarrhea and/or its associated symptoms in the adult non-human animal. According to this aspect, the adult non-human animal is selected from bovine, equine, camel, ovine, swine animals, buffaloes, bison, goats, or camels. In particular embodiments, the animal is an equine animal, such as a horse, or the animal is a camel. In the method, the proanthocyanidin polymer composition can be administered as an enteric coated pharmaceutical composition or as a non-enteric coated pharmaceutical composition.

According to the above method, the proanthocyanidin polymer can be a proanthocyanidin polymer from C. lechleri, such as SB 300, SP 303, crofelemer and pharmaceutically acceptable compositions thereof. In various embodiments of the method, the diarrhea being treated or prevented or the incidence or severity of reduced is secretory or watery diarrhea associated with one or more circumstances selected from conditions during transportation, general anesthesia, surgery, agitation, nervousness, racing, chronic pain, or colitis, including acute colitis. In other embodiments, the non-human animal is additionally suffering from an infection or disease associated with one or more of bacteria, parasites, viruses, or protozoa. In an embodiment, the proanthocyanidin polymer is administered as powder reconstituted with a liquid selected from oral electrolytes, milk, milk replacer, physiological saline, or water; or it is administered as a bolus. In an embodiment, the proanthocyanidin polymer is administered in the animal's feed or drink. In an embodiment, the proanthocyanidin polymer is administered to the animal in an amount of at least 50 mg to 500 mg, or in an amount of 250 mg. In an embodiment, the proanthocyanidin polymer composition is in the form of a gel, paste, or gel paste, which can be administered to the animal by topical application to the roof of the animal's mouth. For topical application, the gel, paste, or gel paste is contained in a delivery device, which can be a syringe. In a particular embodiment, the proanthocyanidin polymer composition or botanical extract derived from C. lechleri is administered in a paste formulation at a dose of 2 mg/kg, where the approximate body weight of an adult animal is on the order of 1000 pounds (lb.)/440 kg.

According to the above method, the gel, paste, or gel paste comprises polymeric microparticles or nanoparticles containing the proanthocyanidin polymer. In an embodiment, the polymeric microparticles or nanoparticles are pH-sensitive. In an embodiment, the symptoms associated with the stress-induced diarrhea in the adult non-human animal and treatable by the method of the invention include dehydration, body weakness and electrolyte loss.

In another of its aspects, the present invention provides a method of treating or preventing or reducing the incidence or severity of stress-induced diarrhea and/or symptoms thereof in an adult non-human animal, e.g., an equine animal (horse) or a camel, the method comprising orally administering to the animal in need thereof a pharmaceutically acceptable composition comprising an aqueous soluble proanthocyanidin polymer from Croton lechleri, wherein the composition is formulated as a gel or paste, and is administered to the animal in an amount of at least 50 mg by topical application to the roof of the mouth, thereby treating, preventing or reducing incidence or severity of the diarrhea and/or the symptoms thereof in the animal. In certain embodiments of the method, the animal is also infected with one or more microorganisms selected from bacteria, viruses, parasites, or protozoa, and the proanthocyanidin polymer composition is administered to the animal in an amount of at least 100 mg. In an embodiment, the proanthocyanidin polymer composition is in the form of a gel contained in a delivery device, which can be a syringe. In certain embodiments, the gel comprises polymeric microparticles or nanoparticles containing the proanthocyanidin composition, and the polymeric microparticles or nanoparticles can be pH-sensitive. According to this method, the proanthocyanidin polymer composition can be administered as an enteric coated or a non-enteric coated pharmaceutical composition. In addition, the proanthocyanidin polymer from C. lechleri can be SB 300, SP 303, crofelemer and pharmaceutically acceptable compositions thereof. In a particular embodiment, the proanthocyanidin polymer is enterically protected beads, including enteric beads comprising SB 300 or SP 303.

In another of its aspects, the present invention provides a method of treating, preventing, reducing the severity or incidence of in an adult equine animal, such as a horse, a diarrhea condition selected from stress-induced diarrhea, Potomac Horse Fever-induced diarrhea, or chronic undifferentiated diarrhea and/or associated symptoms thereof, the method comprising orally administering to an equine animal in need thereof a pharmaceutically acceptable composition comprising an aqueous soluble proanthocyanidin polymer from Croton lechleri, wherein the composition is administered to the equine animal in an effective amount and under conditions for treating, preventing, reducing the incidence or the severity of the diarrhea and/or the associated symptoms in the intestine/gut of the animal. In an embodiment, the proanthocyanidin polymer composition is administered to the equine animal in an amount of 1 to 8 mg/kg for 1 to 15 days. In other embodiments, the proanthocyanidin polymer composition is administered in the feed bag of the animal. The feed bag could also include, but is not limited to, grains, milling by-products, added vitamins, minerals, fats/oils and other energy or nutritional sources. The proanthocyanidin composition could also be provided as an aqueous supplement in the water container/dispenser of the animal. In some embodiments, the proanthocyanidin polymer composition is formulated as a gel or paste, and is administered to the animal in an amount of at least or about 1 to 8 mg/kg by topical application to the roof of the mouth, thereby treating the diarrhea and/or the symptoms thereof in the animal. According to the method, the proanthocyanidin polymer composition can be administered as an enteric coated or as a non-enteric coated pharmaceutical composition and can be SB 300, SP 303, crofelemer and pharmaceutically acceptable compositions thereof.

In an aspect, the invention provides a method of treating, preventing, reducing the incidence or severity in an adult ruminant animal, such as a camel, diarrhea and/or its associated symptoms, the method comprising orally administering to the animal in need thereof a pharmaceutically acceptable composition comprising an aqueous soluble proanthocyanidin polymer from Croton lechleri, wherein the composition is formulated or administered to the animal in an effective amount and under conditions to bypass the animal's rumen so as to treat, prevent or reduce the incidence or severity of the diarrhea and/or its associated symptoms in the intestine/gut of the animal, e.g., a camel. In an embodiment, the animal is afflicted with stress-induced diarrhea, chronic undifferentiated diarrhea, or diarrhea associated with bacterial, viral, parasitic, or protozoan infection. In an embodiment, the proanthocyanidin polymer composition is contained in a container holding greater than 1 gram (g) and is administered to the animal more than once a day for at least one day. In another embodiment, the composition is contained in a 3 gram (g) container and administered to the animal twice a day for 3 days. In another embodiment, the proanthocyanidin polymer composition is administered as a drench to the animal or is administered from a ready-to-use bottle containing the composition. In an embodiment, the proanthocyanidin polymer composition is formulated as a gel or paste, and is administered to the animal in an amount of at least or about 1 mg/kg by topical application to the roof of the mouth. In another embodiment, proanthocyanidin polymer composition is formulated as a powder and administered to the animal as a reconstituted liquid formulation or admixed with feed. In other embodiments, the proanthocyanidin polymer composition is administered in a volume of 20 to 50 liters or in a volume of 25 to 30 liters. In an embodiment, the proanthocyanidin polymer composition is administered as an enteric coated pharmaceutical composition; in another embodiment, the proanthocyanidin polymer composition is administered as an non-enteric coated pharmaceutical composition. According to the method, the proanthocyanidin polymer from C. lechleri can be SB 300, SP 303, crofelemer and pharmaceutically acceptable compositions thereof. In an embodiment, the proanthocyanidin polymer from C. lechleri is preferably enteric protected beads including SB 300 and SP 303.

### DETAILED DESCRIPTION OF THE INVENTION

The methods of the invention are directed to treating the debilitating problem of diarrhea in adult non-human animals. Diarrhea is an all-too common malady affecting the gastrointestinal (GI) tracts of non-human animals, particularly large animals of many types, such as, without limitation, adult horses and camels. In basic terms, the condition occurs when an imbalance of mineral ions in the animal's GI tract alters the normal water balance within the GI tract. This can be caused by a number of conditions and agents, including bacterial, viral, or parasitic infection, electrolyte imbalance, disruption to the hindgut, reduced intestinal flora, malabsorption issues, changes in diet or feed, medication, or ingestion of a toxin.

An alteration in the diet or feed of an animal can be a common cause of diarrhea. By way of example, any change in grain or hay feed, or pasture access, for horses should be made incrementally to avoid introducing gastrointestinal problems. Overfeeding can cause diarrhea, as can feeding to animals poor quality (e.g., spoiled or moldy) grain, feed, or hay. Molds and toxins can form in hay, grain and feed that is not kept dry. Feed containing antibiotics or other additives, e.g., growth hormones, and/or such feed meant for other animals, can also cause diarrhea in certain animals consuming them. Animals can develop allergies or sensitivities to feed ingredients, with diarrhea as a consequence. If animals such as horses or camels eat hay off of sandy ground or graze short stubble in a sandy pasture, sand accumulation (called “sand colic”) in the intestines may lead to partial blockage of the colon and irritation of the intestinal lining, and ultimately cause diarrhea in the animal. At its worst, a horse or camel can accumulate more than 100 pounds of sand in its colon. If the soil upon which an animal such as a horse is sandy, a high likelihood of contracting sand colic exists, as well as the animal's experiencing diarrhea. The methods of the invention can be used to treat diarrhea, including episodic diarrhea, in such affected animals.

Diarrhea that is caused by more than simple digestive upset in adult animals, e.g., horses, camels, cattle, and the like, can result in serious and significant illness. When caused by bacterial or viral infection, intestinal parasitism, intestinal flora imbalance, gastrointestinal or metabolic abnormality, or stress, the animal may be clinically ill and require veterinary attention. By way of example, horses that are ill with diarrhea can present with a number of other symptoms, including colic, dehydration, increased thirst, lack of appetite, depression, weight loss, colitis, pot belly, eye dullness, poor coat and malaise.

Infection such as Salmonellosis can cause symptoms ranging from acute, severe diarrhea to chronic diarrhea, weight loss and death in afflicted adult animals. Animals can also be carriers of Salmonella spp. and remain symptomless; such animals may develop diarrhea upon hospitalization or antibiotic treatment. Infection with Clostridium spp. can cause acute enteritis in afflicted adult animals, and sometimes death. Parasitic infestation, especially equine parasites, such as small strongyles or nematode worms, can cause severe diarrhea and serious illness, particularly in older horses, resulting in marked dehydration and protein loss. Yet another causative agent of diarrhea is associated with Potomac Horse Fever (PHF), particularly in horses in the northeast of the United States. PHF is caused by the organism Neorickettsia risticii, which relies on a parasite living in freshwater snails and aquatic insects as a reservoir for its life cycle and transmission to horses. It is likely that horses become infected with N. risticii by ingesting an aquatic insect or snail while grazing, or consume a flying insect that has landed in drinking water. Other modes of infection include the attraction of insects harboring N. risticii to the barn area by outdoor lights during the night, infection of food and water by the insects, and subsequent ingestion by an animal.

Stress-induced diarrhea, which is particularly treatable by the practice of the methods of the invention, also has numerous causes. For example, stress associated with long and/or frequent transportation of animals, travel in the heat or under other poor conditions, general anesthesia and surgery, or chronic pain may result in colitis or inflammation of the animal's large intestine. Stress induced by excitement or fear in an animal leads to a release of hormones that increase blood flow to the intestines. The severity of the diarrhea caused by such stress relates directly to the amount of stress the animal feels. In addition, exercise that is too intense for the animal's condition, or heavy exercise in extreme heat conditions can induce diarrhea by raising the animal's core temperature to a level at which normal intestinal flora and fauna die, thus causing a flora imbalance or reduction from the norm in the animal. Diarrhea induced by stress in animals needs to be treated, particularly if it does not recede in the animal as stress levels are lowered.

The methods according to the present invention are highly suitable for treating diarrhea in adult animals caused by one or more of the agents and conditions described herein. A preferred embodiment of the invention relates to the treatment of stress-induced diarrhea, particularly in adult animals such as horses and camels, with an effective amount of a polymeric proanthocyanidin composition or extract from Croton spp. under conditions and forms of administration suitable and efficacious for the treatment as described herein. In addition, the methods of the invention provide a solution to the common problem of watery diarrhea, including episodic diarrhea, in adult equine animals and camels. The methods and treatments of the invention improve gastrointestinal/gut health and normalize stool formation in animals suffering from diarrheal conditions, including, by way of example, watery diarrhea in adult horses and camels.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve alleviating one or more symptoms of a disease or condition, although not necessarily all of the symptoms, of the disease or condition, or attenuating the symptoms or progression of the disease or condition. Curing or eliminating the disease or condition from the animal is an optimal outcome of the practice of the methods of the invention. Embodiments of the invention include administering compositions of the invention to prevent or reduce the incidence or severity of diarrheal disease, particularly when non-human animals will be exposed or subjected to events or environments that make them particularly susceptible to diarrhea, such as stress-inducing situations described herein.

According to the invention, treatment of an animal in need thereof typically involves the use or administration of an effective amount or a therapeutically effective amount of a proanthocyanidin polymer or a proanthocyanidin polymer composition preferably from a Croton spp., particularly C. lechleri, provided as either an enteric or non-enteric formulation. Effective amount refers to the quantity (amount) of the composition, and the like, that induces a desired response in the animal subject upon administration or delivery to the animal. Optimally, an effective amount produces a therapeutic effect in the absence of, or with little or virtually no, adverse effects or cytotoxicity in the animal. Alternatively, any adverse effects associated with an effective amount are optimally outweighed by the therapeutic benefit achieved.

The treatment methods are directed to ameliorating, preventing, inhibiting, reversing, attenuating, alleviating, abrogating, minimizing, suppressing, reducing, decreasing, diminishing, stabilizing, eradicating, curing, or eliminating diarrhea and/or its associated symptoms caused by a variety of different agents or environmental factors and influences that adversely affect the health and survivability of adult animals. In an embodiment, the diarrhea is secretory/watery diarrhea. Such diarrhea can be a clinical sign of gastrointestinal (GI) disease in an animal; it can also reflect primary disorders outside of the digestive system, such as disorders affecting the large bowel or the small bowel. The methods described herein are suitable for treating diarrhea resulting from different mechanisms involved in the pathogenesis of the disorders, for example, osmotic diarrhea, secretory diarrhea, episodic diarrhea, or inflammatory and infectious diarrhea in affected animals in need thereof. In an embodiment, the adult animal can suffer from diarrhea associated with inflammation of the lining of the colon, such as colitis, or acute colitis, which can be caused by infection or inflammation of the bowel. In specific embodiments, the proanthocyanidin polymer composition of the invention may be administered to the adult non-human animal to prevent the occurrence or reduce the severity of diarrhea, for example, prior to subjecting the animal to a stressful activity or event such as racing, travel, surgery, anesthesia and the like. The administration of the proanthocyanidin polymer composition may be continued during and even after the stressful activity or even to prevent the occurrence or reduce the severity of diarrhea in the adult, non-human animal.

Proanthocyanidins and Tannins Obtained from Plant Extracts

Proanthocyanidins are types of condensed tannins, which are found in a large number of plants and are classified as hydrolyzable or condensed. Tannins and, in particular, proanthocyanidins are contained in many plants used in traditional medicine as treatment or prophylaxis for diarrhea (See, e.g., Yoshida et al., 1993, Phytochemistry, 32:1033; Yoshida et al., 1992, Chem. Pharm. Bull., 40:1997; Tamaka et al., 1992, Chem. Pharm. Bull., 40:2092).

Proanthocyanidins are comprised of at least two or more monomer units that may be of the same or different monomeric structure. The monomer units (generally termed “leucoanthocyanidins”) are generally monomeric flavonoids which include catechins, epicatechins, gallocatechins, galloepicatechins, flavanols, flavonols, flavan-3,4-diols, leucocyanidins and anthocyanidins. The polymer chains are thus based on different structural units, creating a wide variation of polymeric proanthocyanidins and a large number of possible isomers (Hemingway et al., 1982, J. C. S. Perkin, 1:1217). Larger polymers of the flavonoid 3-ol units are predominant in most plants and often have average molecular weights above 2,000 daltons (Da), containing 6 or more units (Newman et al., 1987, Mag. Res. Chem., 25:118).

Proanthocyanidin polymers and proanthocyanidin are found in a wide variety of plants, especially those having a woody habit of growth (e.g., Croton spp. and Calophyllum spp.). A number of different Croton tree species, including Croton sakutaris, Croton gossypifolius, Croton palanostima, Croton lechleri, Croton erythrochilus and Croton draconoides, which are endemic to South America, produce a red viscous latex sap called Sangre de Drago or “Dragon's Blood”. The red viscous latex is known for its medicinal properties. For example, U.S. Pat. No. 5,211,944 describes the isolation of an aqueous soluble proanthocyanidin polymer composition from Croton spp. (See also, Ubillas et al., 1994, Phytomedicine, 1:77). The isolation of an aqueous soluble proanthocyanidin polymer composition from Calophyllum inophylum is also described in U.S. Pat. No. 5,211,944.

In an embodiment, a proanthocyanidin polymer from C. lechleri, or a composition thereof, is crofelemer. Crofelemer (CAS 148465-45-6) is an oligomeric proanthocyanidin of varying chain lengths derived from the Dragon's Blood of Croton lechleri, a tree of the family Euphorbiaceae, which is sustainably harvested under fair trade work practices in the Amazon. It has an average molecular weight of approximately 1900 Da to approximately 2700 Da. The monomers comprising crofelemer comprise catechin, epicatechin, gallocatechin, and epigallocatechin. The chain length of crofelemer ranges from about 3 to about 30 units with an average chain length of about 8 units. Crofelemer has the chemical formula: (C₁₅O_{6,7}H₁₂)ₙ and a molecular mass of 860-9100 g/mol. The antisecretory mechanism of action of crofelemer involves the targeting and inhibition of two, distinct intestinal chloride channels, namely, the cystic fibrosis transmembrane regulator conductance (CFTR) channel, which is a cAMP-stimulated Cl⁻ channel, and the calcium-activated chloride channel (CaCC), as reported, for example, by Tradtrantip, L et al., 2010, “Crofelemer, an Antisecretory Antidiarrheal Proanthocyanidin Oligomer Extracted from Croton lechleri, Targets Two Distinct Intestinal Chloride Channels”, Mol. Pharmacol., 77(1):69-78). A general structure of crofelemer is shown below. In the structure, an H at the R position of the structure signifies procyanidin; an OH at the R position of the structure signifies prodelphinidin.

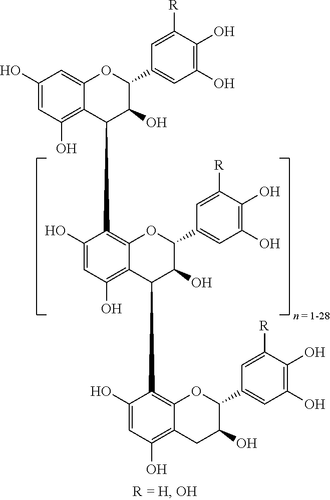

In accordance with an embodiment of the invention, crofelemer, or a pharmaceutically acceptable formulation or composition comprising crofelemer, is employed in the treatment methods as the proanthocyanidin polymer from Croton lechleri.

In an embodiment, SP 303, an oligomeric proanthocyanidin from Croton lechleri, (also known as crofelemer) is the proanthocyanidin polymer from Croton lechleri, or a pharmaceutically acceptable formulation or composition comprising SP 303, which is suitable for use in the treatment methods of the invention. SP-303 (R. Ubillas et al., 1994, Phytomedicine, 1:77-106) is largely composed of purified proanthocyanidin oligomers (−)-galloepicatechin and (+)-gallocatechin, (−)-epicatechin and (+)-catechin and is suitable for use in the enteric and non-enteric formulations and compositions for administration in the treatment methods described herein.

In another embodiment, SB 300, a proanthocyanidin polymer extract from Croton lechleri is the proanthocyanidin polymer from Croton lechleri, or a pharmaceutically acceptable formulation or composition comprising SB 300, which is suitable for use in the treatment methods of the invention. SB 300, as described, for example, by Fischer, H. et al., (2004, J. Ethnopharmacol., 93(2-3):351-357) provides a natural product extract that is particularly amenable for both enteric and non-enteric formulations and compositions, and is highly functional and cost-effective in the treatment methods described herein.

A pharmaceutically acceptable composition comprising a proanthocyanidin polymer from Croton lechleri and employed in the treatment methods of the invention can be obtained from C. lechleri, e.g., as described in WO 00/47062 to Shaman Pharmaceuticals, Inc., the contents of which are incorporated by reference herein, and formulated as a food or dietary supplement or nutraceutical formulation.

In other embodiments, compositions useful in the methods of the invention comprise a raw latex obtained from a Croton species or a Calophyllum species, or an extract obtained from a Croton species or a Calophyllum species, which are not specifically polymeric proanthocyanidin compositions. Exemplary extracts are described in Persinos et al., 1979, J. Pharma. Sci., 68:124 and Sethi, 1977, Canadian J. Pharm. Sci., 12:7.

In an embodiment, the proanthocyanidin polymer from Croton lechleri is formulated with an enteric coating or matrix in a variety of dosage formats known in the art (See, e.g., WO 00/47062 and U.S. Pat. Nos. 7,441,744 and 7,323,195, the contents of which are incorporated herein, and as briefly described below. In another embodiment, the proanthocyanidin polymer is formulation without an enteric coating or matrix. Both enteric and non-enteric forms of the proanthocyanidin polymer from Croton lechleri, for example, SB 300, are intended for use in the methods of the present invention.

### Preparation of Proanthocyanidin Polymer Compositions and Formulations

The proanthocyanidin polymer composition effective for treating secretory diarrhea according to the invention is comprised of monomeric units of leucoanthocyanidins. More particularly, the composition is comprised of proanthocyanidin polymers of 2 to 30 flavonoid units, preferably 2 to 15 flavonoid units, more preferably 2 to 11 flavonoid units and most preferably an average of 7 to 8 flavonoid units with a number average molecular weight of approximately 2500 Da. The proanthocyanidin polymer composition is preferably soluble in an aqueous solution. Preferred for use in the methods according to the invention is a proanthocyanidin polymer from C. lechleri; such a C. lechleri proanthocyanidin polymer may be in the form of a pharmaceutically acceptable composition.

Examples of proanthocyanidin polymeric compositions useful in the present invention are preferably isolated or purified from a Croton spp., namely, Croton lechleri, or Calophyllum spp. by any method known in the art. For example, the proanthocyanidin polymer composition may be isolated from a Croton spp. or Calophylum spp. by the method disclosed in U.S. Pat. No. 5,211,944 or in Ubillas et al. (1994, Phytomedicine, 1:77-106, called SP 303 therein), both of which are incorporated herein by reference. Other isolation methods are described in U.S. Pat. Nos. 7,556,831 and 8,067,041 (Example 2), the contents of which are incorporated by reference herein. PCT application PCT/US00/02687, published as WO 00/47062, the contents of which are incorporated by reference herein, also discloses a method of manufacturing a proanthocyanidin polymeric composition isolated from Croton spp. or Calophyllum spp., and enteric formulations of proanthocyanidin polymer dietary supplements, as well as methods of their preparation. Another illustrative method for isolating proanthocyanidin polymer from C. lechleri (such as crofelemer) is found in U.S. Pat. Nos. 7,341,744 and 7,323,195, the contents of which are expressly incorporated herein. As described above, the SP 303 and SB 300 purified forms of oligomeric proanthocyanidin polymer from Croton lechleri are suitable for use in the treatment methods of the invention.

In an embodiment, the proanthocyanidin polymer composition may be generally isolated by the method following process, such as provided in U.S. Pat. No. 7,341,744. Latex collected from Croton lechleri plants is mixed with purified water (preferably one part latex to two parts purified water). Any insoluble material in the latex solution is allowed to settle, e.g., by leaving the mixture at 4° C. overnight (12 hours). The supernatant is pumped away from the residue and is extracted with a short chain alcohol, such as n-butanol. The extraction is preferably performed multiple times, such as three times. After each extraction, the alcohol phase is discarded and the aqueous phase is retained. The aqueous phase is concentrated, for example, using an ultrafiltration device with a 1 kD cut-off membrane. This membrane can be a low protein binding cellulose membrane, or, alternatively, a polypropylene, teflon or nylon membrane can be used. The membrane used should be compatible with acetone. The purpose of the ultrafiltration is to remove the water from the material.

The retentate from the ultrafiltration is then concentrated to dryness, for example using tray-dryers at approximately 37° C. (±2° C.). The dried material is subsequently dissolved in water and is then chromatographed on a cation exchange column (e.g., a CM-Sepharose column) and a size exclusion column (e.g., an LH-20 column). In the preferred two column system, material is run over a CM-Sepharose and then an LH-20 column in a series. Specifically, the dissolved material is loaded onto the cation exchange column and is then washed with purified water. The proanthocyanidin polymer material is eluted from the cation exchange column with an aqueous acetone solution (preferably 30% acetone), thereby loading the proanthocyanidin polymer material onto the sizing column. The sizing column is disconnected from the cation exchange column and the material is then eluted off of the sizing column with an aqueous acetone solution (preferably 45% acetone). The fractions are collected and monitored with a UV detector, e.g., at a wavelength of 460 nm. Fractions containing the proanthocyanidin polymer material are combined and concentrated, for example, by ultrafiltration using, e.g., a 1 kD cut-off membrane (as described above for the ultrafiltration step prior to the chromatography steps). The retentate may then be concentrated to dryness using a suitable drying method, such as, but not limited to, a rotary evaporator, at a temperature of approximately 37° C. (±2° C.). Other suitable drying methodologies include, but are not limited to, tray drying and spray drying. Example 10 of U.S. Pat. No. 7,341,744 provides additional, non-limiting, methodology for preparing a composition comprising proanthocyanidin polymer, which can be used according to the invention.

### Methods of Treatment and Applications of Use

The invention is directed to methods of treating diarrhea associated with pathogenic infection and non-pathogenic causes, such as stress, particularly in adult animals, comprising administering to an animal in need of such treatment, a proanthocyanidin polymer composition from a Croton species or Calophyllum species in an amount effective to treat the diarrhea. In preferred embodiments, the proanthocyanidin polymer is from a Croton species, namely, Croton lechleri. Treating the diarrhea can involve reducing the severity and duration of the diarrhea in the animal. Treating the diarrhea can also involve increasing the survivability of the animal undergoing treatment. In an embodiment, the diarrhea is secretory or watery diarrhea. The invention also provides methods of preventing, including reducing the incidence or severity of, diarrhea in non-human animals, particularly prior to being exposed to events or an environment that might increase the risk of diarrheal disease.

The methods of the invention relate to the treatment of non-human animals, notably but not limited to, adult livestock, farm animals, and domestically or commercially used animals. Often, but not always, the animals are large in size and have complex gastrointestinal systems. For example, in horses, which are optimally suited for the diarrhea treatment methods of the invention, the intestinal volume of the animal is large, with the main site of diarrhea being in the colon. Consequently, the described methods provide treatment of such animals with a proanthocyanidin polymer from C. lechleri, or composition thereof, providing for an adequate amount and appropriate distribution of the proanthocyanidin polymer in the gut of the animal so as to treat the diarrhea and/or its symptoms and optimally cure the diarrhea in the animal. In a related manner, ruminant animals, such as camels, which are also optimally suited for the diarrhea treatment methods of the invention, possess multi-chambered stomachs, e.g. four stomach compartments, including a rumen or first compartment of the alimentary canal, which serves as the primary site for microbial fermentation of ingested feed. The described methods provide treatment of ruminant animals such as camels with a proanthocyanidin polymer from C. lechleri, or composition thereof, providing for an adequate amount and appropriate distribution of the proanthocyanidin polymer in the intestinal tract of the animal, rather than the rumen, so as to treat the diarrhea and/or its symptoms and optimally cure the diarrhea.

In accordance with an embodiment of the diarrhea treatment methods of the invention, the proanthocyanidin polymer from C. lechleri is orally administered to animals having a large intestinal volume and/or multi-compartment stomachs and a rumen in a large volume and/or high concentration to treat the diarrhea. For example, the C. lechleri proanthocyanidin polymer, or composition thereof, is orally administered to the animal in a volume of about 10 to 50 liters, or in a volume of 20 to 40 liters or in a volume of about 25 to 30 liters, so as to target and reach the animal's large intestine/gut for optimal treatment. In an embodiment in which ruminant animals are treated for diarrhea in accordance with the methods of the invention, the C. lechleri proanthocyanidin polymer, or composition thereof, is formulated for oral administration to the animal such that the C. lechleri proanthocyanidin polymer bypasses the rumen and reaches the intestine for delivery of the composition to the affected area and to treat the diarrhea more efficiently. In adult ruminants there is a need to bypass the rumen to avoid dilution of the product in a large volume of liquid. The ruminants have a specific anatomical structure, called the esophageal groove, in the stomach that closes and forms a tube which enables liquid to bypass the rumen. The closing of this esophageal groove is controlled by neural stimulation from suckling. Thus, by way of example, bypassing the rumen may be achieved by using a bottle to administer the product.

The types of non-human adult animals for which the treatment methods are suitable are not particularly limited as to animal type, genus, or species. In general, adult farm animals, food-source animals, livestock animals, animals bred or kept for various purposes, such as sport (e.g., racing, riding), transport, domestic, companion, industrial uses (e.g., hauling, pulling, plowing), and the like, are particularly amenable to treatment according to the methods of the invention. For example, encompassed by the methods of the invention is the treatment of cows, cattle, steer, camels, sheep, rams, horses, pigs, goats, bison, buffalo, llamas, donkeys, mules, yaks, etc. Adult, exotic animals, such as zoo animals, are also embraced by the treatments of the invention. The treatment of diarrhea, such as stress-induced diarrhea, in adult equines (horses) and camels is particularly embraced by the described methods.

In accordance with the described methods, the C. lechleri proanthocyanidin polymer composition reduces chloride flux across intestinal epithelial cells and reduces fluid movement into the intestinal lumen, which results in fluid loss and dehydration associated with secretory diarrhea. Therefore, the pharmaceutical formulations and methods of the invention are useful in prophylactic and therapeutic applications in the treatment of secretory diarrhea, especially in preventing the dehydration and electrolyte loss that accompanies secretory/watery diarrhea.

In a particular embodiment, the methods of the invention treat diarrhea resulting from infection by the Salmonella spp. microorganism with an effective amount of C. lechleri proanthocyanidin polymer, or composition thereof, or with a latex, extract, or food supplement botanical extract derived therefrom. The treatment of diarrhea caused by Salmonella spp. with C. lechleri proanthocyanidin polymer, or a composition thereof, or with a latex, extract or food supplement botanical extract derived therefrom, is an unexpected and surprising aspect of the invention, because Salmonella spp. cause diarrhea by a mechanism of action and by affecting cellular pathways and responses that are distinct and different from the mechanism of action associated with the activity and function of the C. lechleri proanthocyanidin polymer.

More specifically, mechanism of action of the C. lechleri proanthocyanidin polymer, e.g., crofelemer or SB 300, or a composition thereof, is through the inhibition of both the cystic fibrosis transmembrane conductance regulator protein (CFTR) chloride ion channel and the calcium-activated chloride ion channels (CaCC). The polymeric proanthocyanidin composition acts by blocking chloride ion channel secretion and the accompanying high volume water loss occurring in diarrhea, thus normalizing the flow of chloride ions and water in the gastrointestinal (GI) tract. However, Salmonella microorganisms trigger diarrhea in infected hosts by producing several virulence factors. One such factor is a protein called SopE, which is injected into intestinal epithelium cells where it triggers a cascade of intracellular signaling events once the bacteria enter the GI tract. (See, e.g., S. Zhang et al., 2003, Infection and Immunity, 71(1):1-12; and A. J. Mueller et al., 2009, Cell Host and Microbe, 6(2):125-136). The binding of the SopE protein to two specific GTPase proteins alters the cell membrane and allows the bacteria to penetrate the cell. In addition, the two GTPase proteins activate Caspase-1 inside the cell, which is a key factor in inflammatory responses. Caspase-1, in turn, causes the production of proinflammatory mediators (cytokines) that attract macrophages which phagocytize the bacteria that has penetrated into the intestinal tissue and cells; however, Salmonella spp. bacteria remaining in the intestinal lumen are not seriously affected. The heightened immune response that exists in the infected animals as a consequence of the infection results in serious inflammation, fluid accumulation and distress for the host animal.

Because Salmonella, which causes a disease pathology and an inflammatory immune response that lead to diarrhea without significantly affecting the CTRF or CaCC, it is considered quite surprising and unexpected that a proanthocyanidin polymer composition which functions by inhibiting these channels is effective in treating diarrhea induced by the Salmonella microorganism. However, the treatment of diarrhea in Salmonella-infected adult animals, such as, e.g., horses and camels, with a proanthocyanidin polymer composition (e.g., SB-300) according to present methods demonstrates an unpredicted effectiveness of the composition against diarrhea resulting from a source associated with a different etiology.

In an embodiment, the animals treated by the methods of the invention are greater than four months of age. In a related embodiment, the animals have fully developed and competent GI tracts and colon function. In an embodiment, the animal has a weight (mass) of about 400 to 800 kg. In an embodiment, the animal has a weight of about 500 to 700 kg. In an embodiment, the animal has a weight of about 550 to 650 kg. In an embodiment, the animal has a weight of about 600 kg. In an embodiment, the animals have undifferentiated diarrhea of stress-induced origin.

The methods of the invention encompass treatment of diarrhea resulting from Potomac Horse Fever (PHF), in which affected equine animals are orally administered a pharmaceutically acceptable composition comprising an aqueous soluble proanthocyanidin polymer isolated from a Croton species or a Calophyllum species, wherein the composition is administered to the animal in an effective amount and under conditions for treating the diarrhea and/or its associated symptoms in the animal's intestine (gut). The various modes and routes of administering the composition as described herein are suitable for the treatment method.

### Physiologically and Pharmaceutically Acceptable Formulations

The proanthocyanidin polymer from C. lechleri, or a composition thereof, can be provided in any physiologically, pharmaceutically, or therapeutically acceptable form. The pharmaceutical composition can be formulated for oral administration as, illustratively, but without limitation, powders; crystals; granules; small particles, including microparticles; particles sized on the order of micrometers, e.g., microspheres and microcapsules; particles sized on the order of millimeters, particles sized on the order of nanometers, e.g., nanoparticles; beads; microbeads; pellets; pills; tablets; microtablets; compressed tablets or tablet triturates; molded tablets or tablet triturates; and in capsules, which are either hard or soft and contain the composition as a powder, particle, bead, solution or suspension. The pharmaceutical composition can also be formulated for oral administration as a solution or suspension in an aqueous liquid, as a liquid incorporated into a gel capsule, as a gel, as a paste or gel paste, or as any other convenient formulation for administration. The composition can be formulated for rectal administration, as a suppository, enema or other convenient form. The proanthocyanidin polymeric composition can also be provided as a controlled release system (See, e.g., Langer, 1990, Science 249: 1527-1533). The composition can be formulated as a dietary supplement or food supplement, e.g., as described in WO 00/47062, for administration to an animal in need thereof according to the present invention.

In a particular embodiment, a formulation or composition comprising a botanical extract derived from C. lechleri, SB 300, or SP 303, is provided in the form of a gel or paste formulation that is orally administered to the adult animal, in need, twice daily for three days, preferably, three consecutive days. In a particular embodiment, the twice daily doses are administered to the animal twelve hours apart. The paste formulation is particularly suitable as a product that acts locally in the gut and is minimally absorbed systemically. The paste product specifically addresses the normalization of stool formation and ion and water flow in the intestinal lumen of the treated animals and does not alter gastrointestinal motility, i.e., is not constipating. As but one mode of oral delivery, the paste formulation can be placed in the roof of the animal's mouth. In a particular embodiment, the paste formulation comprises beads (nano or microparticles) comprising enterically coated SB 300 or SP 303 and is orally administered to the adult animals. In an embodiment, the paste comprising SB 300 enteric beads is orally administered to an animal, such as a horse, twice daily for three days. In some embodiments, the paste is orally administered for three consecutive days. In an embodiment, the paste comprising SB 300 enteric beads is orally administered to an animal, e.g., a horse, in need at a dose of 2 mg/kg twice daily for three days. The formulation is especially suitable for the normalization of stool formation in a short time period, e.g., less than a week or less than two weeks; for mitigation of weight loss; and reduction in supportive care costs, rehydration therapies, such as oral rehydration, in animals undergoing treatment and afflicted with diarrhea.

The pharmaceutical formulation can also include any type of pharmaceutically acceptable excipients, additives, carriers, or vehicles. By way of nonlimiting example, diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose can be added to the proanthocyanidin polymer composition to increase the bulk of the composition. In addition, binders, such as, but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, can be added to the formulation to increase its cohesive qualities. Further, lubricants, such as, but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate and magnesium lauryl sulfate can be added to the formulation. Also, glidants, such as, but not limited to, colloidal silicon dioxide or talc can be added to improve the flow characteristics of a powdered formulation. Disintegrants, such as, but not limited to, starches, clays, celluloses, algins, gums, crosslinked polymers (e.g., croscarmelose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch can also be added to facilitate disintegration of the formulation in the intestine.

In some embodiments, the pharmaceutical formulations contain the proanthocyanidin polymer composition with an enteric coating, in addition to another pharmaceutically acceptable vehicle. In an embodiment, the proanthocyanidin polymer composition can be directly-compressed into a tablet. The tablet can be without excipients and of pharmaceutically acceptable hardness and friability, optionally, with a lubricant, e.g., without limitation, magnesium stearate, and enteric coated. In another embodiment, the pharmaceutical compositions containing the proanthocyanidin polymer composition alternatively include one or more substances that either neutralize stomach acid and/or enzymes or are active to prevent secretion of stomach acid. These formulations can be prepared by methods known in the art (See, e.g., methods described in Remington's “The Science and Practice of Pharmacy,” 22nd Edition, Editor-in-Chief: Lloyd V. Allen, Jr., Pharmaceutical Press, Royal Pharmaceutical Society, London, U K, 2013; and U.S. Pat. No. 7,323,195).

In an embodiment, the proanthocyanidin polymer composition is formulated with a substance that protects the proanthocyanidin polymer and/or the polymer composition from the stomach environment. For such protection, the proanthocyanidin polymer composition can be enteric coated. Enteric coatings are those coatings that remain intact in the stomach, but will dissolve and release the contents of the dosage form once it reaches the small intestine. A large number of enteric coatings are prepared with ingredients that have acidic groups such that, at the very low pH present in the stomach, i.e. pH 1.5 to 2.5, the acidic groups are not ionized and the coating remains in an undissociated, insoluble form. At higher pH levels, such as in the environment of the intestine, the enteric coating is converted to an ionized form, which can be dissolved to release the proanthocyanidin polymer composition. Other enteric coatings remain intact until they are degraded by enzymes in the small intestine, and others break apart after a defined exposure to moisture, such that the coatings remain intact until after passage into the small intestines. A variety of polymers are useful for the preparation of enteric coatings, and the application of an enteric coating to the proanthocyanidin polymer composition can be accomplished by any method known in the art for applying enteric coatings, as may be found, for example, and without limitation, in U.S. Pat. Nos. 7,323,195 and 7,341,744, incorporated herein by reference.

In another embodiment, the pharmaceutically acceptable composition of the proanthocyanidin polymer composition is formulated as enteric coated granules or powder (microspheres with a diameter of 300-500 microns) provided in either hard shell gelatin capsules or suspended in an oral solution for pediatric administration. The enteric coated proanthocyanidin polymer composition powder or granules can also be mixed with food, particularly for administration to neonatal or young animals. Such preparations may be prepared using techniques well known in the art. In addition, the proanthocyanidin polymer composition granules and powder can be prepared using any method known in the art, such as, but not limited to, crystallization, spray-drying or any method of comminution, preferably using a high speed mixer/granulator, as described, for example and without limitation, in U.S. Pat. No. 7,323,195, incorporated herein by reference.

In other embodiments, the proanthocyanidin polymer composition is in the form of an aqueous suspension in admixture with suitable excipients. Non-limiting examples of excipients that are suitable for the manufacture of aqueous suspension include suspending agents, for example, methylcellulose, sodium carboxymethylcellulose, hydroxy-propylmethylcellulose, sodium alginate, polyvinyl-pyrrolidone, gum tragacanth and gum acacia; dispersing or wetting agents, which may be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, e.g., sucrose, saccharin or aspartame.

Dispersible powders and granules suitable for the preparation of an aqueous suspension by the addition of water provide the proanthocyanidin polymer composition in admixture with a dispersing or wetting agent, suspending agent and one or more preservatives. Suitable dispersing or wetting agents and suspending agents are exemplified by those stated above. Additional excipients, for example, sweetening, flavoring and coloring agents, may also be present.

In an embodiment, the proanthocyanidin polymer composition is a gel or gel formulation. In an embodiment, the proanthocyanidin polymer composition is a paste formulation. In an embodiment, the paste formulation contains a purified botanical extract derived from C. lechleri. In another embodiment, the paste formulation contains enterically coated beads comprising SB 300 or SP 303. In an embodiment, the paste formulation contains enteric protected SB 300 beads. In an embodiment, the gel or paste is contained or preloaded in a delivery device, such as a syringe, e.g., a needle-less syringe, or other type of applicator or delivery system, especially for oral delivery. A gel or paste formulation is particularly suited for administration to Salmonella spp.-infected neonatal and young foals, but also is applicable for other Salmonella spp.-infected adult and neonatal animals, such as those described herein. In an embodiment, the gel or paste is not contained in a delivery device, but is administered to the roof of the mouth of the animal, particularly one that is too incapacitated or ill to eat or drink, thereby eschewing an oral or other mode of administration. In an embodiment, the gel or paste comprises pH-sensitive polymeric particles, such as microparticles or nanoparticles, to allow for pH-dependent uptake of the active compound into cells and/or the pH-dependent release of the active compound in different pH environments in an animal. Processes for generating granules and particles comprising the proanthocyanidin polymer composition or a compressible form thereof are as known and practiced in the art, and as provided, for example, in U.S. Pat. No. 7,341,744, the contents of which are incorporated by reference herein. In an embodiment, gels are prepared for oral delivery and contain copolymers, such as poloxamers and Pluronics of different types, e.g., Pluronic F.

In another embodiment, the proanthocyanidin polymer composition is in a paste formulation, preferably for oral administration. For example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent.

Oily suspensions may be formulated by suspending the C. lechleri proanthocyanidin polymer as active ingredient in a vegetable oil, e.g., arachis oil, olive oil, sesame oil or coconut oil, or in mineral oil, such as liquid paraffin. The oily suspensions may contain a thickening agent, e.g., beeswax, hard paraffin or cetyl alcohol. Oral preparations can include sweetening agents as mentioned above and flavoring agents to improve palatability. Pharmaceutically acceptable preservatives, for example, an anti-oxidant such as ascorbic acid, can also be added to such compositions.

The C. lechleri proanthocyanidin polymer pharmaceutical compositions used in the methods of the invention may also be in the form of an oil-in-water emulsions. The oily phase may be a vegetable oil such as olive oil or arachis oil, or a mineral oil such as liquid paraffin or mixtures of these oils. Examples of emulsifying agents include, without limitation, naturally-occurring phosphatides, e.g., soy bean, lecithin, and esters or partial esters derived from fatty acids and hexitol anhydrides, e.g., sorbitan monooleate, and condensation products of partial esters with ethylene oxide, e.g., polyoxyethylene sorbitan monooleate. Sweetening, coloring and flavoring agents can be included in the emulsions.

Syrups and elixirs containing the C. lechleri proanthocyanidin polymer may also can be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, orally deliverable or administrable aqueous or oleagenous suspension. This suspension may be formulated according to methods known in the art using suitable dispersing or wetting agents and suspending agents, such as those mentioned above. The sterile pharmaceutical preparation may also be a sterile solution or suspension in a non-toxic parenterally-acceptable diluent or solvent, for example, a solution in 1,3-butane diol. Illustrative, acceptable vehicles and solvents that may be used in the preparations include water, Ringer's solution and isotonic sodium chloride solution. Co-solvents, e.g., ethanol, propylene glycol or polyethylene glycols, may also be included. In addition, sterile, fixed oils, e.g., any bland, fixed oil such as synthetic mono- or diglycerides, are conventionally employed as solvents or suspending media and may be used. In addition, fatty acids, such as oleic acid and the like, may be used in injectable preparations.

### Dosage Forms and Administration

In a particular embodiment for treating diarrhea or preventing in adult animals, e.g., without limitation, horses, camels, llamas, cows, etc., the proanthocyanidin polymer composition is in powder, e.g., reconstitutable powder, form. The composition may be enterically coated or not enterically coated. In an embodiment, the non-human animal is of an age that its large intestine/bowel and colon are fully competent and functional. In an embodiment, the non-human animal is greater than four months of age. In an embodiment, the non-human animals are adult horses or camels. In an embodiment, the animals are afflicted with or at risk for stress-induced secretory diarrhea. In an embodiment, in addition to, or rather than, stress-induced diarrhea, the animal experiences involvement of one or more of a bacterial, e.g., Salmonella spp. or Clostridium spp., infection, a viral infection, e.g., rotavirus and/or coronavirus, whose mechanism of action involves infection and subsequent destruction of the cells lining the intestinal tract, or a parasitic infection, e.g., nematodes.

In an embodiment, the powder form of the proanthocyanidin polymer composition used for treatment is reconstituted or mixed with liquid, such as oral electrolytes, milk or a milk replacer/substitute, water, physiological saline, to produce a liquid form or suspension. The proanthocyanidin polymer composition is generally 5 to 30% of the suspension. Those skilled in the art will appreciate that due to the higher purity of compositions such as SP-303 or crofelemer and SB-300, more by weight of SB-300 than SP-303 will need to be used in formulations to achieve the same amount of the active ingredient of the proanthocyanidin polymer composition. SB-300 generally has about 67% by weight of the proanthocyanidin polymer composition while SP-303 has higher purity, for example 99-100%. The composition is administered at a dosage of 1 to 8 mg/kg, preferably 2 to 4 mg/kg. In an embodiment, the powder form of the proanthocyanidin polymer composition is provided in the form of individual dosages in packets, e.g., packaged dosage forms, wherein some number of individual packets are provided for use in a treatment regimen. Such packaged dosage forms could contain the proanthocyanidin polymer composition in an amount of 1 to 8 g or 1 to 8 mg/kg packaged in an amount for a particular adult animal. The number of individual doses that can be packaged and provided together is not intended to be limiting, and can include, for example, one to twenty packaged doses; one to ten packaged doses; two, four, six, eight, ten, or more packaged doses, as well as numbers of packaged doses in-between the foregoing, for efficiency of use, handling and for commercial efficacy. In an embodiment particularly applicable to large animals, especially those having large intestines and rumens, the volume of reconstituted powder for administration is necessarily large, for example, greater than 20 liters, so as to reach the desired site in the animal's intestine/gut.

In another embodiment, the powder form of the proanthocyanidin polymer composition is provided in a container, such as a bag, box, bucket, or pail (e.g., 5 lb. to 25 lb. pails), in which the powder can be in an amount of, for example, 100 grams (g) or more, and can optionally include a measuring device, such as a scoop, cup, spoon, trowel, dipper, or ladle. Such containers encompass, for example, an individual daily dose of the proanthocyanidin polymer composition; or an amount suitable for multiple treatments, e.g., a two-day treatment, three-day treatment, four day treatment, etc. An effective amount of the powder can also be mixed with feed for consumption by the animals in need thereof. In certain embodiments, the proanthocyanidin polymer composition is administered at a dosage of 1 to 8 mg/kg once or twice a day for a duration of from 1 to 30 days.

In an embodiment, the proanthocyanidin polymer composition is administered or delivered to an adult animal afflicted with or at risk for diarrhea and in need thereof by providing the compound as a bolus or pill. In an embodiment, the proanthocyanidin polymer composition formulated as bolus, e.g., a pill, capsule, or tablet, is orally administered to an adult animal afflicted with diarrhea and/or symptoms thereof, e.g., dehydration, electrolyte imbalance, or at risk for diarrhea, directly in the mouth. In an embodiment, the bolus can be a large dose given orally or intravenously, with orally preferred. In an particular embodiment, the treatment regimen comprises administering a dose of 250 mg to 550 mg of the product for a smaller animal, such as a dog, and 1 to 8 grams or 1 to 8 mg/kg for larger adult animals such as horses, cows and camels, e.g., as embraced by one bolus per animal for a determined time period, for example, for one, two, or three or more days. The product can be provided to an animal in need thereof in portions of the complete dose, in which the portions are administered one or two or more times per day. Alternatively, the complete dose can be administered to an animal in need thereof one or two or more times per day. In a particular embodiment, the treatment encompasses the administration of the C. lechleri proanthocyanidin polymer composition two times a day for consecutive days, e.g., greater than two days to seven days.

In an embodiment, the proanthocyanidin polymer composition is in a gel or gel formulation. In an embodiment, the gel is contained or preloaded in a delivery device, such as a syringe or other type of injector or delivery system, especially for oral delivery. In an embodiment, the gel comprises pH-sensitive polymeric particles, such as microparticles or nanoparticles, to allow for pH-dependent uptake of the active compound into cells and/or the pH-dependent release of the active compound in different pH environments in an animal. A gel formulation is particularly suited for administration to adult horses and camels, but also is applicable for other adult animals, such as those described herein. In an embodiment, the gel is not contained in a delivery device, but is administered to the roof of the mouth of the animal, particularly one that is too incapacitated or ill to eat or drink, thereby eschewing an oral or other mode of administration. In an embodiment, gels are prepared for oral delivery and contain copolymers, such as poloxamers and Pluronics of different types, e.g., Pluronic F. Processes for generating granules and particles comprising the proanthocyanidin polymer composition or a compressible form thereof are as known and practiced in the art, and as provided, for example, in U.S. Pat. No. 7,341,744, the contents of which are incorporated by reference herein.

In another embodiment, the proanthocyanidin polymer composition is in a paste formulation, preferably for oral administration. For example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent. In an embodiment, the paste formulation contains beads with enterically coated SB 300 or SP 303, which is administered to an animal, such as a horse, at a dose of 2 mg/kg. More particularly, the paste formulation containing enterically coated SB 300 beads is administered to a horse at a dose of 2 mg/kg, twice a day for three days. In an embodiment, the paste containing enteric protected SB 300 beads is administered twice a day at twelve hour intervals.

The routes of administration of the C. lechleri proanthocyanidin polymer to afflicted animals are not intended to be limiting. Illustratively, administration can be via any suitable, convenient or preferred route of administration including oral, buccal, dental, periodontal, via food source (animal feed), nutrition source, or libation source, otic, inhalation, endocervical, intramuscular, subcutaneous, intradermal, intracranial, intralymphatic, intraocular, intraperitoneal, intrapleural, intrathecal, intratracheal, intrauterine, intravascular, intravenous, intravesical, intranasal, ophthalmic, biliary perfusion, cardiac perfusion, spinal, sublingual, topical, transdermal, intravaginal, rectal, ureteral, or urethral. In certain embodiments, oral, buccal, and food and/or drink supplement are particularly suitable routes. In an embodiment, the product is an aqueous formulation and is provided to the animal as a drench or directly from a ready-to-use (RTU) bottle directed to the esophageal cavity so as to more effectively reach the animal's intestine/gut for optimal activity. In a related embodiment, administration can also be by inclusion in the regular or special diet of the animal, such as in a functional food for the animals or companion animals.

Dosage forms can include, without limitation, oral, injectable, transdermal, aerosol including metered aerosol, chewable products or pellets, capsules, capsule containing coated particles, nanoparticles, or pellets, capsule containing delayed release particles, capsule containing extended release particles, concentrates, creams and augmented creams, suppository creams, discs, dressings, elixirs, emulsions, enemas, extended release films or fibers, gases, gels, metered gels, granules, delayed release granules, effervescent granules, implants, inhalants, injectable lipid complexes, injectable liposomes, inserts or devices, extended release inserts, intrauterine devices, jellys, liquids, extended release liquids, lotions, augmented lotions, oils, ointments, augmented ointments, pastes, pastilles, pellets, powders, reconstituted powders, extended release powders, metered powders, solutions, drops, concentrated solutions, gel forming solutions/drops, sponges, sprays, metered sprays, suppositories, suspensions, suspensions/drops, extended release suspensions, syrups, tablets/pills, chewable tablets/pills, tablets/pills containing coated particles, delayed release tablets/pills, dispersible tablets/pills, effervescent tablets/pills, extended release tablets/pills, orally disintegrating tablets/pills, tapes, or troches/lozenges. The dosages can be provided as formulations, compositions, pharmaceutically acceptable formulations and compositions, physiologically acceptable formulations and compositions, including pharmaceutically and physiologically acceptable carrier, excipients, diluents, or vehicles as known and used in the art.

For oral administration, the C. lechleri proanthocyanidin polymer, including a C. lechleri botanical extract, or a composition thereof, is preferably encapsulated and formulated with suitable carriers, and the like, in solid dosage forms. Nonlimiting examples of suitable carriers, excipients, diluents and vehicles include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, edible oils, and the like. The formulations can also include lubricating agents, wetting agents, emulsifying and suspending agents, preserving agents, sweetening agents or flavoring agents. The compositions can be formulated to provide rapid, sustained, extended, or delayed release of the active ingredient after administration to the animal by employing protocols and methods well known in the art. The formulations can also include compounds or substances that reduce proteolytic degradation and promote absorption such as, for example, surface active agents.

As will be appreciated by those having skill in the art, the specific dose can be calculated according to the approximate body weight, body mass, or body surface area of the animal, or the volume of body space or mass to be occupied. The dose also depends on the particular route of administration selected by the practitioner. Further refinement of the calculations necessary to determine an appropriate dosage for treatment is routinely made by those of ordinary skill in the art, for example, using appropriate assays and analytical procedures, such as has been described for certain compounds (e.g., Howitz et al., Nature, 425:191-196, 2003). Exact dosages can be determined based on standard dose-response studies. Therapeutically effective doses for treatment of afflicted animals can be determined, by titrating the amount of the active product given to the animal to arrive at the desired therapeutic effect, while minimizing side effects. For use in treating diarrhea, such as secretory or watery diarrhea, and its symptoms in adult animals in accordance with the methods of the invention, a therapeutically acceptable form of the C. lechleri proanthocyanidin polymer, or a composition thereof, is administered, particularly orally administered, in an amount ranging from 0.1 to 100 mg/kg per day. In other embodiments, the amount can range from about 0.1 to about 10 mg/kg/day; or from about 0.1 to about 25 mg/kg/day, or from about 0.1 to about 30 mg/kg/day, or from about 0.1 to about 40 mg/kg/day. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, etc., as well as incremental dose amounts in between. In still other embodiments, the amount can range from about 1 to about 10 mg/kg/day once, twice or more daily; or from about 1 to about 5 mg/kg/day, from about 1 to about 8 mg/kg/day, from about 1 to about 10 mg/kg/day, or from about 2 to about 4 mg/kg/day once, twice or more daily. In an embodiment, the amount of the C. lechleri proanthocyanidin polymer composition for administration is 2 mg/kg two times a day. In an embodiment, the 2 mg/kg dose is administered twice a day for three days. In a more particular embodiment, SB 300 enteric beads are formulated in a paste which is administered to a neonatal or young animal, e.g. a horse foal, at a dose of 2 mg/kg two times a day for three days. In other embodiments, the foregoing amounts of the C. lechleri proanthocyanidin polymer composition are administered, for example, twice daily, three times daily, four times daily, or more than four times daily, rather than once per day. Higher doses, e.g., 50 mg/kg or 100 mg/kg per day or twice or more daily, may be required, as necessary, to treat diarrhea and accompanying dehydration in animals in need.

In other embodiments, a suitable dose for the C. lechleri proanthocyanidin polymer may range from about 1 mg/day to about 1000 mg/day. In an embodiment, a suitable dose may range from about 10 mg/day to about 500 mg/day. In an embodiment, a suitable dose may range from about 50 mg/day to about 500 mg/day. In an embodiment, a suitable dose may range from about 50 mg/day to about 350 mg/day. In an embodiment, a suitable dose may range from about 100 mg/day to about 250 mg/day. It will be understood that the ranges include the lower and higher amounts specified, as well as incremental dose amounts in between.

In some embodiments, daily doses, including multiple daily doses, e.g., twice or three times a day, of the C. lechleri proanthocyanidin polymer product may be 2 mg, 5 mg, 10 mg, 15 mg, 20 mg, 25 mg, 50 mg., 100 mg, 150 mg, 175 mg, 200 mg, 225 mg, 250 mg, 275 mg, 300 mg, 325 mg, 350 mg, 375 mg, 400 mg, 500 mg (or there between) per animal. Administration schedules may also be altered to achieve a therapeutically effective concentration of the C. lechleri proanthocyanidin polymer to treat or prevent the diarrhea and its symptoms as described herein. In some embodiments, the compound may be administered once per day, twice per day, thrice per day, 4 times per day, 5 times per day, 7 times per day or 10 times per day. Often the dosage is divided into equal parts administered throughout the day, however in some embodiments related to treating more severe or entrenched symptoms, it may be useful to tailor the dosage administration schedule so that most of the daily treatment is administered at a predetermined time of the day, e.g., the beginning half of the day. In some embodiments, about 50% 60%, 70% or 80% of the dosage is administered in the first half of the day. In other embodiments, it may be more appropriate to administer most of the dosage in the latter half of the day so that about 50%, 60%, 70% or 80% of the dosage is administered in the latter half of the day.

In other embodiments, for the treatment methods, a suitable dose for the C. lechleri proanthocyanidin polymer product, or the C. lechleri proanthocyanidin polymer composition, such as SP 303 or SB 300, may range from about 1 mg to about 1000 mg, either daily or multiple times per day. In an embodiment, a suitable dose may range from about 10 mg to about 500 mg, either daily or multiple times per day. In an embodiment, a suitable dose may range from about 50 mg to about 350 mg, either daily or multiple times per day. In an embodiment, a suitable dose may range from about 30 mg to about 400 mg, either daily or multiple times per day. In an embodiment, a suitable dose may range from about 100 mg to about 250 mg, either daily or multiple times per day. In an embodiment, a suitable dose may range from about 50 mg to about 300 mg, either daily or multiple times per day. It will be understood that the ranges include the lower and higher amounts specified, as well as amounts in between. The doses administered multiple times per day can be given for consecutive days, e.g., two days, three days, four days, five days, six, days, seven days, or more, in some embodiments. A dose administered multiple times per day may embrace two, three, four, five, six, or more times per day. Other dosing schedules, such as every other day, or every third day, every fourth day, etc. are embraced by the invention. In an embodiment, the amount of the C. lechleri proanthocyanidin polymer composition is 2 mg/kg two times a day for three days. In a more particular embodiment, enteric protected SB 300 beads are formulated in a paste which is administered to an adult animal, such as a horse, at a dose of 2 mg/kg two times a day for three days. In addition, one having skill in the art will appreciate that doses and amounts administered to the animal can vary, given the wide range of weights of the animals undergoing treatment, as well as the animal species and type of digestive system, e.g., ruminant or non-ruminant. In an embodiment the C. lechleri proanthocyanidin polymer is SB 300. In an embodiment the C. lechleri proanthocyanidin polymer is enterically coated SB 300. In an embodiment the C. lechleri proanthocyanidin polymer is non-enterically coated SB 300.

It will be understood that the dose amount actually administered can be determined by the practitioner, in the light of the relevant circumstances, including the severity of the condition or symptoms thereof being treated, the form of the product to be administered, the age, weight, and response of the individual animal receiving treatment, as well as the chosen route of administration.

The methods of the invention further embrace the administration of pharmaceutically acceptable formulations of the proanthocyanidin polymer composition either alone or in combination with other supplements or agents for treatment or amelioration of the symptoms of secretory diarrhea, such as rehydration agents, electrolytes (e.g., sodium, potassium, magnesium, chloride and formulations thereof), antibiotics, gut-lining protectants, such as kaolin, pectin, or bismuth liquid, and fluid adsorbents, such as attapulgite. Other agents may include anti-motility agents, although because many of the microorganisms and pathogens that are associated with diarrhea induction in adult animals concomitantly decrease gut motility, the use of anti-motility drugs may be contraindicated. Natural biological products, e.g., Lactobacillus, Bifidobacterium, or Streptococcus faecium, other bacteria and yeast microorganisms, or probiotics, may also be employed as additives to restore the natural balance of intestinal flora in the affected neonatal animals. Such natural biological products, e.g., probiotics as known in the art, may be administered in conjunction with the C. lechleri proanthocyanidin polymer or composition thereof, for example, prior to, at the same time as, or after the administration of the proanthocyanidin polymer or composition to a non-human animal. In addition, a reconstituted C. lechleri proanthocyanidin polymer or composition thereof may include probiotics in accordance with the present invention.

### Specific Embodiments Encompassed by the Invention

The present invention is further directed to uses and methods encompassed by the following embodiments.

In an embodiment, the present invention provides the use of a pharmaceutical composition comprising an aqueous soluble proanthocyanidin polymer from Croton lechleri for treating an adult non-human animal having stress-induced diarrhea, wherein said composition is orally administered to an animal in need thereof in an amount effective to treat said stress-induced diarrhea. In an embodiment, the adult non-human animal is selected from the group consisting of bovines, equines, camels, ovines, swine, buffaloes, bison, or goats. More specifically, the adult non-human animal is an equine animal or a camel. In an embodiment, the C. lechleri proanthocyanidin polymer composition is administered as an enteric coated pharmaceutical composition or as a non-enteric coated pharmaceutical composition. In an embodiment, C. lechleri proanthocyanidin polymer is selected from the group consisting of SB 300, SP 303, and crofelemer. In other embodiments, the C. lechleri proanthocyanidin polymer composition is administered in an amount of (i) at least 50 mg to 500 mg; (ii) at least 250 mg; (iii) 1 to 8 mg/kg for 3 days; (iii) 1 to 8 mg/kg once or twice a day for 3 to 30 days; (iv) 1 to 4 mg/kg once or twice a day for 15 to 30 days; (v) 2 mg/kg twice a day for 3 days; or (vi) 2 mg/kg per day two times per day for two or more consecutive days. In an embodiment, the diarrhea is secretory or watery diarrhea associated with one or more circumstances selected from conditions during transportation, general anesthesia, surgery, agitation, nervousness, racing, chronic pain, or colitis; and optionally wherein the non-human animal is additionally suffering from an infection or disease associated with one or more of bacteria, parasites, viruses, or protozoa. In other embodiments, the C. lechleri proanthocyanidin polymer is administered (i) as a powder reconstituted with a liquid selected from oral electrolytes, milk, milk replacer, physiological saline, or water; (ii) as a bolus; (iii) in animal feed or drink; or (iv) in the form of a gel, paste, or gel paste. In an embodiment, the gel, paste, or gel paste is administered to the animal by topical application to the roof of the animal's mouth. In an embodiment, the gel, paste, or gel paste is contained in a delivery device, which is optionally a syringe. In an embodiment, the gel, paste, or gel paste comprises polymeric microparticles or nanoparticles containing the C. lechleri proanthocyanidin polymer; wherein the polymeric microparticles or nanoparticles are optionally pH-sensitive. In an embodiment, the adult non-human animal has stress-induced diarrhea and the aqueous soluble proanthocyanidin polymer from Croton lechleri is formulated as a gel or paste, said gel or paste formulation administered orally in a therapeutically effective amount by topical application to the roof of the mouth. In an embodiment, the C. lechleri proanthocyanidin polymer composition is administered to the animal in an amount of at least 10 mg. In an embodiment, the gel or paste comprises polymeric microparticles or nanoparticles containing the C. lechleri proanthocyanidin composition. In an embodiment, the polymeric microparticles or nanoparticles are pH-sensitive. In an embodiment, the paste formulation comprises enteric coated SB 300 micro or nanoparticles. In an embodiment, the non-human animal is an adult equine animal having a diarrhea condition selected from stress-induced diarrhea, Potomac Horse Fever-induced diarrhea, or chronic undifferentiated diarrhea. In an embodiment, the C. lechleri proanthocyanidin polymer composition is administered as an aqueous supplement in the water container/dispenser of the animal.

In another embodiment, the present invention provides the use of an aqueous soluble proanthocyanidin polymer from Croton lechleri for treating an adult ruminant animal having diarrhea, said treatment comprising orally administering said aqueous soluble proanthocyanidin polymer from Croton lechleri to said adult ruminant animal in need thereof under conditions to bypass the animal's rumen and in an effective amount to treat the diarrhea in the intestine of the animal. In an embodiment related to this use, the ruminant animal is a camel. In an embodiment, the diarrhea is selected from stress-induced diarrhea, chronic undifferentiated diarrhea, or diarrhea associated with bacterial, viral, parasitic, or protozoan infection. In an embodiment, the C. lechleri proanthocyanidin polymer is held for administration (i) in a container holding greater than 1 gram (g) and is administered more than once a day for at least one day; or (ii) in a 3 g container and is administered to the animal twice a day for 3 days. In an embodiment, the C. lechleri proanthocyanidin polymer is administered to the animal as a drench or from a ready-to-use bottle. In an embodiment, the C. lechleri proanthocyanidin polymer is formulated as a gel or paste, and is administered to the animal in an amount of at least 10 mg by topical application to the roof of the mouth. In an embodiment, the C. lechleri proanthocyanidin polymer is formulated as a powder and administered to the animal as a reconstituted liquid formulation or admixed with feed. In an embodiment, the C. lechleri proanthocyanidin polymer is administered in a volume of 20 to 50 liters or in a volume of 25 to 30 liters. In an embodiment, the C. lechleri proanthocyanidin polymer is administered as an enteric coated pharmaceutical composition or as a non-enteric coated pharmaceutical composition. In an embodiment, the C. lechleri proanthocyanidin polymer is selected from the group consisting of SB 300, SP 303, or crofelemer. In a particular embodiment, the non-human adult animal is an adult equine animal with diarrhea; the C. lechleri proanthocyanidin polymer is a pharmaceutical composition formulated as a paste and orally administered to the animal in an amount of 2 mg/kg per day two times per day for two or more consecutive days. In an embodiment, the paste is applied to the roof of the animal's mouth. In an embodiment, the paste is administered two times a day, twelve hours apart, for three consecutive days; wherein the paste is optionally contained in a delivery device, which can be a syringe. In an embodiment, the C. lechleri proanthocyanidin polymer is selected from the group consisting of SB 300, SP 303, and crofelemer. In an embodiment, the SB 300 is optionally in the form of enteric coated micro or nanoparticles.

The following examples describe the invention in its various aspects and are not intended to be limiting.

### EXAMPLES

### Example 1

### Treatment of Stress-Induced Diarrhea in Adult Camels

Camels can contract diarrhea for many reasons, particularly while in the hospital or at the racetrack, including infectious pathogens, diet changes, medications, exposure to other sick animals and stress. Bacterial enteritis caused by overgrowth of organisms such as Salmonella and Clostridium, as well as antimicrobial drug administration are among the common causes of diarrheal disease in adult camels. Notwithstanding, the mechanisms leading to diarrhea, e.g., electrolyte imbalance and fluid loss, are similar and cause the adverse effect of increased intestinal secretion.

Although fluid therapy may be generally used in most cases of diarrhea in adult camels, an effective antisecretory therapy targeting the camel intestine provides an advantageous approach to the treatment of diarrhea in these animals. According to the present invention, a proanthocyanidin polymer composition as described herein provides beneficial and efficacious treatment of secretory diarrhea adult animals. It regulates chloride and fluid secretion into the intestine by modulating the cystic fibrosis transmembrane regulator (CFTR) and calcium-activated chloride channels (CaCC) in the intestinal epithelium. The regulation of these intestinal chloride channels normalizes flow of chloride ions and associated water into the GI tract. Specifically, the proanthocyanidin polymer or a composition thereof normalizes the cyclic adenosine monophosphate (cAMP) activated CFTR and calcium-activated CaCC chloride intestinal channels; it is not absorbed but acts locally in the gastrointestinal tract.

### Animals

The animals treated in the study include male and female adult camels of greater than one year of age. Only animals treated for intestinal parasites (helminths) and showing signs of acute diarrhea are enrolled in the study. Any animals having chronic diarrhea lasting for more than 3 days are not enrolled in the study.

### Treatment Groups

Two groups of animals undergo testing in this study, as follows:

Group 1—In this group, animals are treated with study agent, C. lechleri proanthocyanidin polymer. Twenty (20) adult camels receive 2 grams of the C. lechleri proanthocyanidin polymer, i.e., approximately 3 grams of SB 300 or 2 grams of crofelemer, as a food supplement in feed bags. The treated camels receive one 3 gram bag of the proanthocyanidin polymer composition twice a day for 3 days, in addition to “standard of care” (SOC) treatment, including fluid and antibiotics. 3 grams of the SB-300 can be diluted in a small volume of water and administered via a bottle, if required.
Group 2—In this control group, ten (10) adult camels receive only SOC treatment, including fluids and antibiotics.
Standard of care: The therapeutic goals can be divided in 3 categories: specific antimicrobial therapy, maintenance of physiological homeostasis of water and electrolyte balance and supportive care for diarrhea. Metronidazole (15 mg/kg orally every 8 hours) is generally considered the treatment of choice. Fluid therapy should be aimed at intravascular and total body water volume replacement (colloid and crystalloid fluids). Aggressive intravenous polyionic fluid therapy should be instituted immediately in animal with enteritis or colitis and the replacement fluid may be administered rapidly (up to 6 to 10 L/hour for a camel or a horse). Because of gastrointestinal losses and serum albumin catabolism many animals experiencing acute colitis or enteritis are hypoproteinemic. Commercial colloids such as plasma, dextran 40, dextran 70 or hydroxyethyl starch (up to 10 ml/kg/day) may be used. Oral intestinal protectants such as bismuth subsalicylate, activated charcoal or di-tri-octahedral smectite (Biosponge®) can be used to reduce toxin uptake through the permeable bowel lining. Restoration of the microbial flora in the large intestine may be useful in the management of animals with gastroenteritis. Saccharomyces boulardii has been used in horses and other adult animals to reduce and the severity and duration of acute enterocolitis.

Treatment group allocation is in chronological order: the two first animals presented at the clinic and showing signs of diarrhea are treated (from Group 1), the third is a control (from Group 2) The procedure for enrollment is continued until 30 camels are enrolled in the study. The animals are examined twice a day by a licensed veterinarian for 3 days and the observations are recorded on the data sheet.

### Parameters to be Tested

The parameters tested in the study involve fecal examination, fecal consistency, volume of diarrhea, frequency of diarrhea and time to resolution of diarrhea, using the criteria presented in the table below.

TABLE 1

| Descriptors | Clinical Finding Score |
|---|---|
| Consistency of diarrhea | |
| Normal, formed feces | 1 |
| Soft feces but formed | 2 |
| Loose feces, sitting on shavings | 3 |
| Watery feces | 4 |
| Volume of diarrhea | |
| Low volume per episode of diarrhea | 1 |
| High volume per episode of diarrhea | 2 |
| Frequency of diarrhea | |
| Low frequency, none or one episode every | 1 |
| 6 hours | |
| Medium frequency, two to three episodes per | 2 |
| 6 hours | |
| High frequency, more than three episodes per | 3 |
| 6 hours | |

In addition, the animals are tested for the presence of Salmonella in the feces whenever possible.

### Study Schedule for Individual Animals

The study schedule for assessing individual animals undergoing evaluation is as follows:

Day 1 Morning Diagnosis
Clinical scoring
Treatment
Afternoon Clinical scoring
Treatment

Day 2 Morning Clinical scoring
Treatment
Afternoon Clinical scoring
Treatment

Day 3: Morning Clinical scoring
Treatment
Afternoon Clinical scoring
Treatment

End of study for the individual animal

A typical clinical data sheet used in the study is presented below.

### Example 2

A representative paste composition of the present invention, which comprises enterically coated SB 300 beads, is presented in this Example. For administration to animals and as noted hereinabove, the paste containing enteric protected SB 300 beads may be contained in a syringe. A paste containing enteric coated SB 300 beads may contain the following components:

| Component | % w/w | Theoretical mg/syringe |
|---|---|---|
| SB 300 enteric beads | 21.91 | 3286.6* |
| Vegetable oil | 64.42 | 9663.5 |
| Cetyl alcohol | 9.76 | 1464.2 |
| Apple flavor | 0.08 | 11.7 |
| Silicon dioxide | 2.73 | 410.0 |
| Butylated hydroxytoluene | 0.04 | 5.9 |
| Titanium dioxide | 1.05 | 158.1 |
| Total | 100.0 | 15000 |

*3286.6 mg SB 300 enteric beads corresponds to 880 mg theoretical SB 300.

All patents, patent applications and publications referred to or cited herein are hereby incorporated by reference in their entireties for all purposes.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

1. A method of treating an adult non-human animal having stress-induced diarrhea, the method comprising orally administering to an animal in need thereof an aqueous soluble proanthocyanidin polymer from Croton lechleri in an amount effective to treat said stress-induced diarrhea.

2. The method according to claim 1, wherein the adult non-human animal is selected from the group consisting of bovines, equines, camels, ovines, swine, buffaloes, bison, or goats.

3. The method according to claim 1, wherein the adult non-human animal is an equine animal.

4. The method according to claim 1, wherein the adult non-human animal is a camel.

5. The method according to claim 1, wherein the C. lechleri proanthocyanidin polymer composition is administered as an enteric coated pharmaceutical composition.

6. The method according to claim 1, wherein the C. lechleri proanthocyanidin polymer composition is administered as a non-enteric coated pharmaceutical composition.

7. The method according to claim 1, wherein the C. lechleri proanthocyanidin polymer is selected from the group consisting of SB 300, SP 303, and crofelemer.

8. The method according to claim 1, wherein the C. lechleri proanthocyanidin polymer composition is administered 1 to 8 mg/kg for 3 days or 1 to 4 mg/kg once or twice a day for 15 to 30 days.

9. The method according to claim 1, wherein the diarrhea is secretory or watery diarrhea associated with one or more circumstances selected from conditions during transportation, general anesthesia, surgery, agitation, nervousness, racing, chronic pain, or colitis.

10. The method according to claim 1, wherein the non-human animal is additionally suffering from an infection or disease associated with one or more of bacteria, parasites, viruses, or protozoa.

11. The method according to claim 1, wherein the C. lechleri proanthocyanidin polymer is administered as powder reconstituted with a liquid selected from oral electrolytes, milk, milk replacer, physiological saline, or water.

12. The method according to claim 1, wherein the C. lechleri proanthocyanidin polymer is administered as a bolus.

13. The method according to claim 1, wherein
the C. lechleri proanthocyanidin polymer is administered in animal feed or drink.

14. The method according to claim 1, wherein the C. lechleri proanthocyanidin polymer composition is in the form of a gel, paste, or gel paste.

15. The method according to claim 14, wherein the gel, paste, or gel paste is administered to the animal by topical application to the roof of the animal's mouth.

16-17. (canceled)

18. The method according to claim 14, wherein the gel, paste, or gel paste comprises polymeric microparticles or nanoparticles containing the C. lechleri proanthocyanidin polymer.

19. The method according to claim 18, wherein the polymeric microparticles or nanoparticles are pH-sensitive.

20. The method according to claim 1, wherein the C. lechleri proanthocyanidin polymer is administered to the animal in an amount of at least 50 mg to 500 mg.

21. The method according to claim 1, wherein the C. lechleri proanthocyanidin polymer is administered to the animal in an amount of at least 250 mg.

22. The method according to claim 1, wherein the symptoms associated with the stress-induced diarrhea in the adult non-human animal include dehydration, body weakness and electrolyte loss.

23-33. (canceled)

34. The method according to claim 15, wherein the paste formulation comprises enteric coated SB 300 beads.

35. (canceled)

36. A method of treating an adult equine animal having a diarrhea condition selected from stress-induced diarrhea, Potomac Horse Fever-induced diarrhea, or chronic undifferentiated diarrhea, the method comprising orally administering to an equine animal in need thereof an aqueous soluble proanthocyanidin polymer from Croton lechleri in an amount effective to treat the diarrhea condition.

37-41. (canceled)

42. The method according to claim 36, wherein the C. lechleri proanthocyanidin polymer composition is administered as an aqueous supplement in the water container/dispenser of the animal.

43. The method according to claim 36, wherein the C. lechleri proanthocyanidin polymer is formulated as a gel or paste, and is administered to the animal in an amount of at least 1 to 8 mg/kg by topical application to the roof of the mouth.

44. The method according to claim 36, wherein the C. lechleri proanthocyanidin polymer is administered as an enteric coated pharmaceutical composition.

45. The method according to claim 36, wherein the C. lechleri proanthocyanidin polymer is administered as an non-enteric coated pharmaceutical composition.

46. A method of treating an adult ruminant animal having diarrhea, the method comprising orally administering to the adult ruminant animal in need thereof an aqueous soluble proanthocyanidin polymer from Croton lechleri under conditions to bypass the animal's rumen and in an amount effective to treat the diarrhea in the intestine of the animal.

47. The method according to claim 46, wherein the ruminant animal is a camel.

48. The method according to claim 46, wherein the diarrhea is selected from stress-induced diarrhea, chronic undifferentiated diarrhea, or diarrhea associated with bacterial, viral, parasitic, or protozoan infection.

49-50. (canceled)

51. The method according to claim 46, wherein the C. lechleri proanthocyanidin polymer is administered as a drench to the animal.

52-66. (canceled)

67. The method according to claim 1, wherein the C. lechleri proanthocyanidin polymer or composition thereof is administered in conjunction with probiotics.
